(19) United States
(12) Patent Application Publication
(10) Pub. No.: US 2023/0260106 A1
(43) Pub. Date: Aug. 17, 2023

(54) DETECTING ROBUSTNESS OF MACHINE LEARNING MODELS IN CLINICAL WORKFLOWS

(71) Applicant: Siemens Healthcare GmbH, Erlangen (DE)

(72) Inventors: Costin Florian Ciusdel, Azuga (RO); Saikiran Rapaka, Pennington, NJ (US); Lucian Mihai Itu, Brasov (RO); Puneet Sharma, Princeton Junction, NJ (US)

(21) Appl. No.: 17/650,669

(22) Filed: Feb. 11, 2022

(51) Int. Cl. G06T 7/00; G16H 30/40; G16H 50/20; G06T 7/11

(52) U.S. Cl. CPC G06T 7/0012; G16H 30/40; G16H 50/20; G06T 7/11; G06T 2207/20081; G06T 2207/20084; G06T 2207/10081

(57) ABSTRACT

Systems and methods for determining a robustness of a machine learning based medical analysis network for performing a medical analysis task on input medical data are provided. Input medical data is received. Results of a medical analysis task performed based on the input medical data using a machine learning based medical analysis network are received. A robustness of the machine learning based medical analysis network for performing the medical analysis task is determined based on the input medical data and the results of the medical analysis task using a machine learning based audit network. The determination of the robustness of the machine learning based medical analysis network is output.

### TECHNICAL FIELD

The present invention relates generally to machine learning models in clinical workflows, and in particular to detecting robustness of machine learning models in clinical workflows.

### BACKGROUND

Machine learning models have been applied to perform various medical analysis tasks, such as, e.g., detection, segmentation, quantification, etc. Supervised machine learning models are typically trained offline and deployed at a clinical site (e.g., on a medical imaging scanner) or in the cloud for integration into clinical workflows for clinical decision making (e.g., diagnosis, treatment planning, etc.). Such machine learning models are typically trained on a large training dataset to cover a wide range of variations to ensure robust performance. However, regardless of the size of the training dataset, it is likely that such machine learning models will be asked to perform a prediction on datasets that are significantly different from their training dataset

### BRIEF SUMMARY OF THE INVENTION

In accordance with one or more embodiments, systems and methods for determining a robustness of a machine learning based medical analysis network for performing a medical analysis task on input medical data are provided. Input medical data is received. Results of a medical analysis task performed based on the input medical data using a machine learning based medical analysis network are received. A robustness of the machine learning based medical analysis network for performing the medical analysis task is determined based on the input medical data and the results of the medical analysis task using a machine learning based audit network. The determination of the robustness of the machine learning based medical analysis network is output.

In one embodiment, in response to determining that the machine learning based medical analysis network is not robust, it is determined that the machine learning based medical analysis network is not robust due to the input medical data being out-of-distribution with respect to training data on which the machine learning based medical analysis network was trained or due to an artifact in at least one of the input medical data or the results of the medical analysis task. In another embodiment, in response to determining that the machine learning based medical analysis network is not robust, the machine learning based medical analysis network and the machine learning based audit network are retrained based on the input medical data. In another embodiment, in response to determining that the machine learning based medical analysis network is not robust, one or more alternate results of the medical analysis task from other machine learning based medical analysis networks are presented.

In one embodiment, user input editing the results of the medical analysis task to generate final results of the medical analysis task is received. The robustness of the machine learning based medical analysis network is determined based on the final results of the medical analysis tasks.

In one embodiment, the machine learning based audit network is implemented using a normalizing flows model.

In one embodiment, in response to determining that the machine learning based medical analysis network is not robust, an alert to a user notifying the user that the machine learning based medical analysis network is not robust or requesting input from the user is generated. The input may be received from the user overriding the determination that the machine learning based medical analysis network is not robust or editing the results of the medical analysis task.

In one embodiment, the medical analysis task comprises at least one of segmentation, determining centerlines of vessels, or computing a fractional flow reserve (FFR).

These and other advantages of the invention will be apparent to those of ordinary skill in the art by reference to the following detailed description and the accompanying drawings.

### BRIEF DESCRIPTION OF THE DRAWINGS

FIG. 1 shows a method for determining a robustness of a medical analysis network for performing a medical imaging analysis task on input medical data, in accordance with one or more embodiments;

FIG. 2 shows a workflow for training and applying a machine learning based medical analysis network for performing a medical analysis task on input medical data and a machine learning based audit network for determining a robustness of the medical analysis network for performing the medical imaging analysis task on the input medical data, in accordance with one or more embodiments;

FIG. 3 shows a workflow for evaluating user input received for performing a medical analysis task, in accordance with one or more embodiments;

FIG. 4 shows a workflow for calculating FFR (fractional flow reserve) from a CCTA (coronary CT angiography) image, in accordance with one or more embodiments;

FIG. 5 shows graphs comparing robustness determined for independent input datasets in accordance with embodiments described herein with ground truth labels;

FIG. 6 shows an exemplary Glow-style normalizing flows network architecture for an audit network, in accordance with one or more embodiments;

FIG. 7 shows a table for implementing a Glow-style normalizing flows network, in accordance with one or more embodiments;

FIG. 8 shows a table for implementing a two-headed 3D CNN (convolutional neural network) for implementing a scaling function and a translation function, in accordance with one or more embodiments;

FIG. 9 shows a table for implementing a CNN for computing a kernel k and a vector b, in accordance with one or more embodiments;

FIG. 10 shows a network architecture for implementing a normalizing flows audit network, in accordance with one or more embodiments;

FIG. 11 shows a table for implementing the network architecture of FIG. 10, in accordance with one or more embodiments;

FIG. 12 shows training images used for training the normalizing flows audit network, in accordance with one or more embodiments;

FIG. 13 shows a graph showing probability variation across one vessel segment of 80 cross-sections from a test dataset, in accordance with one or more embodiments;

FIG. 14 shows saliency plots for a normalizing flows audit model, in accordance with one or more embodiments;

FIG. 15 shows a workflow for reducing the number of rejected cases in a clinical center, in accordance with one or more embodiments;

FIG. 16 shows an exemplary artificial neural network that may be used to implement one or more embodiments;

FIG. 17 shows a convolutional neural network that may be used to implement one or more embodiments; and

FIG. 18 shows a high-level block diagram of a computer that may be used to implement one or more embodiments.

### DETAILED DESCRIPTION

The present invention generally relates to methods and systems for detecting robustness of machine learning models in clinical workflows. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system.

A machine learning based medical analysis network (or model) may be applied to perform a variety of medical analysis tasks, such as, e.g., detection, segmentation, quantification, clinical decision making, etc. on input medical data. In accordance with embodiments described herein, a machine learning based audit network is provided to evaluate the robustness of the medical analysis network for performing the medical analysis task on the input medical data. The robustness of the medical analysis network refers to the ability of the medical analysis network to accurately perform the medical analysis task on the input medical data. The medical analysis network may not be robust for performing the medical analysis task on the input medical data where, for example, the input medical data is out-of-distribution with respect to the training dataset on which the medical analysis network is trained or where the input medical data comprises artifacts. Advantageously, embodiments described herein enable input medical data, that is or may be input into the medical analysis network, to be flagged where the medical analysis network is not robust for performing the medical analysis task for the input medical data. User input may be requested from a user, or the user may be warned that the prediction of the medical analysis network cannot be trusted for such flagged medical input data.

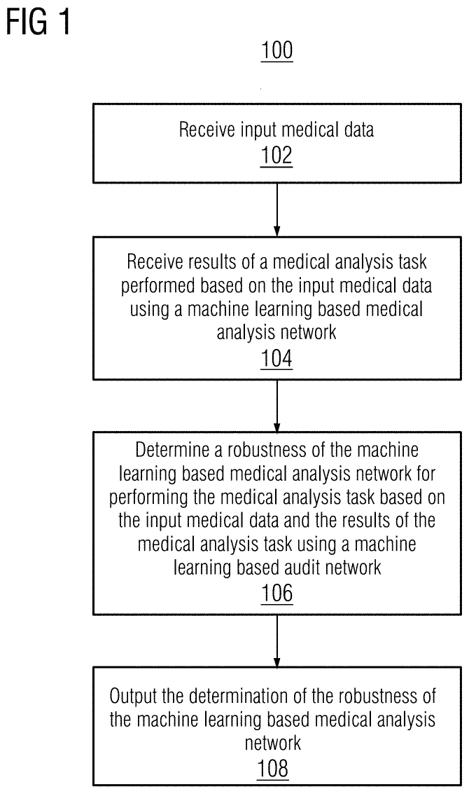

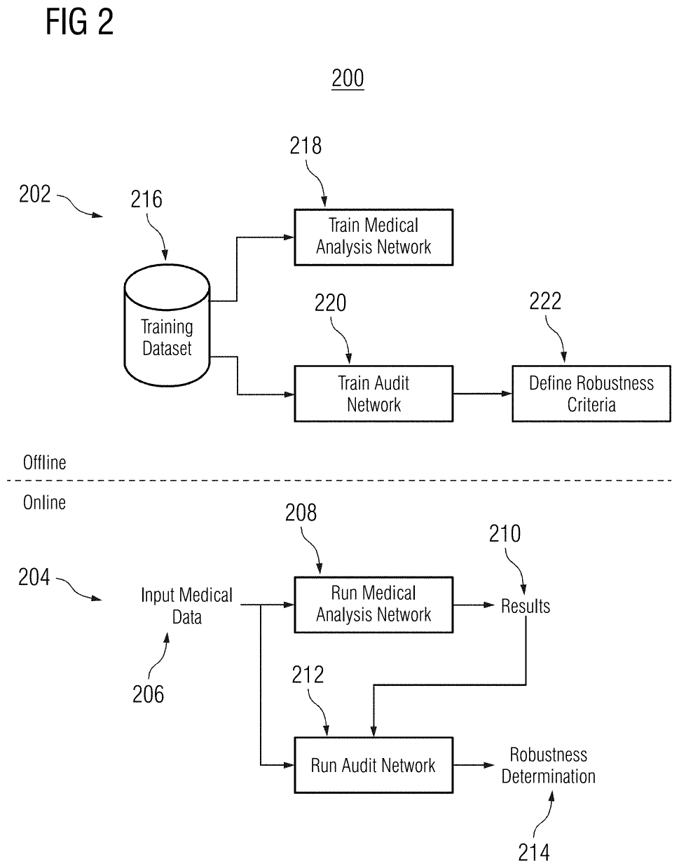

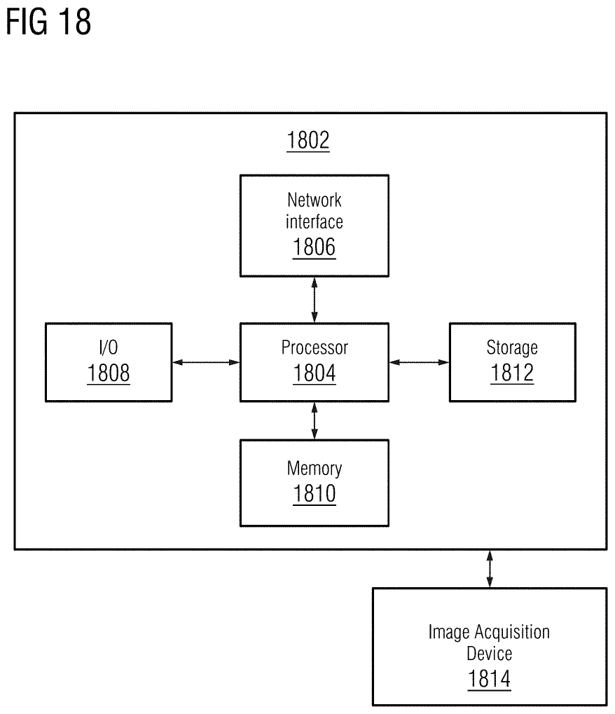

FIG. 1 shows a method 100 for determining a robustness of a medical analysis network for performing a medical imaging analysis task on input medical data, in accordance with one or more embodiments. The steps of method 100 may be performed by one or more suitable computing devices, such as, e.g., computer 1802 of FIG. 18. FIG. 2 shows a workflow 200 for training and applying a machine learning based medical analysis network for performing a medical analysis task on input medical data and a machine learning based audit network for determining a robustness of the medical analysis network for performing the medical imaging analysis task on the input medical data, in accordance with one or more embodiments. FIG. 1 and FIG. 2 will be described together. Workflow 200 of FIG. 2 shows an offline stage 202 for training a medical analysis network and an audit network and an online stage 204 for applying the trained medical analysis network and the trained audit network. In one example, steps of method 100 of FIG. 1 are performed during online stage 204 of FIG. 2.

At step 102 of FIG. 1, input medical data is received. In one example, as shown in FIG. 2, the input medical data may be input medical data 206 of workflow 200. The input medical data may comprise any suitable medical data of a patient.

In one embodiment, the input medical data may comprise input medical images of the patient. The input medical images may be of any suitable modality, such as, e.g., CT (computed tomography), MRI (magnetic resonance imaging), ultrasound, x-ray, or any other medical imaging modality or combinations of medical imaging modalities. The input medical images may be 2D (two dimensional) images and/or 3D (three dimensional) volumes, and may comprise a single image or a plurality of images.

The input medical data may comprise any other suitable medical data of the patient. For example, the input medical data may comprise sensor data acquired from medical sensors on or in the patient, medical forms relating to the patient (e.g., patient questionnaires), or any other medical data of the patient. In one embodiment, the input medical data comprises data output from an upstream machine learning based network, e.g., that performed an upstream medical analysis task in a cascaded workflow.

The input medical data may be received by loading previously acquired medical data from a storage or memory of a computer system or receiving medical data that has been transmitted from a remote computer system. Where the input medical data comprises input medical images, the medical images may be received directly from an image acquisition device (e.g., image acquisition device 1814 of FIG. 18), such as, e.g., a CT scanner, as the medical images are acquired.

At step 104 of FIG. 1, results of a medical analysis task performed based on the input medical data using a machine learning based medical analysis network are received. In one example, as shown in FIG. 2, the results of the medical analysis task may be results 210 from running the medical analysis network at block 208 based on input medical data 206. The medical analysis task may be any suitable medical analysis task, such as, e.g., diagnosis, treatment planning, etc. In one embodiment, the medical analysis task is a medical imaging analysis task such as, e.g., detection, quantification, segmentation, etc. performed based on input medical images. The medical analysis network may be implemented according to any suitable machine learning based architecture. In one embodiment, the medical analysis network is a supervised machine learning model.

At step 106 of FIG. 1, a robustness of the machine learning based medical analysis network for performing the medical analysis task is determined based on the input medical data and the results of the medical analysis task using a machine learning based audit network. In one example, as shown in FIG. 2, the determination of the robustness of the machine learning based medical analysis network is robustness determination 214 determined by running the audit network at block 212 based on input medical data 206 and results 210. The audit network receives the input medical data and the results of the medical analysis task as input and generates a robustness determination as output. The audit network may be implemented according to any suitable machine learning based architecture. In one embodiment, the audit network is an unsupervised machine learning model.

The robustness of the medical analysis network refers to the ability of the medical analysis network to accurately perform the medical analysis task on the input medical data. The medical analysis network may not be robust for performing the medical analysis task on the input medical data where, for example, the input medical data is out-of-distribution with respect to the training dataset on which the medical analysis network is trained (i.e., the input medical data falls outside of the data distribution with respect to the training dataset) or where the input medical data and/or the results of the medical analysis task comprises artifacts. The artifacts may be due to faulty data acquisition, due to the output of a preceding algorithm (e.g., a pre-processing algorithm that generated an incorrect output), faulty user input, etc.

The determination of the robustness of the medical analysis network may be represented in any suitable form. In one embodiment, the determination of the robustness comprises a binary output indicating that the medical analysis network is robust or not robust or that the results of the medical analysis task should be accepted or not accepted. In another embodiment, the determination of the robustness comprises a plurality of classifications. For example, the determination of the robustness may comprise a multi-class output indicating that 1) the medical analysis network is robust (e.g., the output of the medical analysis network can be trusted without user interaction), 2) user feedback is requested (e.g., the output of the medical analysis network should be verified by a user), or 3) the medical analysis network is not robust (e.g., the output of the medical analysis network cannot be trusted). In classification 3, a user may verify the output of the medical analysis network and overrule the determination of the audit network. In a further embodiment, the determination of the robustness comprises a continuous output, where the robustness is represented in a continuous range. For example, the determination of the robustness may be a robustness score representing a measure of dissimilarity of the input medical data from the training data on which the medical analysis network is trained. One or more thresholds may be applied to the robustness score to generate a binary output or a multi-class output.

At step 108 of FIG. 1, the determination of the robustness of the machine learning based medical analysis network is output. For example, the determination of the robustness can be output by displaying the determination of the robustness on a display device of a computer system, storing the determination of the robustness on a memory or storage of a computer system, or by transmitting the determination of the robustness to a remote computer system.

In one embodiment, step 104 of method 100 of FIG. 1 is not performed and the determination of the robustness of the machine learning based medical analysis network at step 106 of method 100 is based on the input medical data but not based on the results of the medical analysis task.

In one embodiment, in response to determining that the medical analysis network is not robust for performing the medical analysis task on the input medical data, an alert may be generated to, e.g., notify the user that the medical analysis network is not robust and/or for requesting user input from the user. In response to the alert, user input may be received from the user to, e.g., override the determination of the audit network, edit the results of the medical analysis task, confirm the determination of the audit network, etc.

In one embodiment, in response to determining that the medical analysis network is not robust for performing the medical analysis task on the input medical data, it may be further determined whether the medical analysis network is not robust due to the input medical data being out-of-distribution with respect to training data on which the medical analysis network is trained or due to the input medical data comprising an artifact. The determination of whether the medical analysis network is not robust due to the input medical data being out-of-distribution or due to the input medical data comprising an artifact may be automatically performed, manually performed, or semi-automatically performed. The automatic determination may be performed using a separate machine learning model or a rule-based approach. The manual determination may be performed by a user labelling the input medical data as being out-of-distribution or as having artifacts. The semi-automated determination may be performed as a combination of the automatic determination and the manual determination.

The medical analysis network and the audit network are trained during a prior offline or training stage. For example, as shown in FIG. 2, the medical analysis network and the audit network may be trained during offline stage 202 to train medical analysis network at block 218 and audit network at block 220 based on training dataset 216. Training dataset 216 may comprise training medical images with labels annotating ground truth results of the medical analysis task. Once the audit network is trained, a robustness criteria may be defined at block 222. For example, the robustness criteria may define one or more thresholds to be applied to the output of the audit network to define a binary output of, e.g., robust or not robust or a multi-class output of, e.g., robust, user feedback required, or not robust.

In one embodiment, where input medical data 206 is determined to be out-of-distribution with respect to training data on which the medical analysis network is trained, the input medical data 206 may be added to update the training dataset 216 and the medical analysis network and the audit network may be retrained based on the updated training dataset 216. In some embodiments, data augmentation techniques may be applied on such out-of-distribution input medical data 206 using, e.g., standard augmentation techniques or by generating synthetic data resembling such input medical data.

In one embodiment, the audit network is trained as a normalizing flows model. A normalizing flows model is a bijective generative model based on deep neural networks. The normalizing flows model utilizes stacks of coupling layers (or stages). At each layer, some inputs are passed through unchanged (Equation 1) while other inputs are modified based on the passed-through inputs in an invertible fashion (Equation 2). The affine coupling may be defined as followings:

y_{0 . . . k}=u_{0 . . . k} (Equation 1)

y_{k+1 . . . m}=u_{k+1 . . . m}s(u_{0 . . . k})t(u_{0 . . . k}) (Equation 2)

where u denotes the input to each layer, y denotes the output of each layer, and k is an index indicating a split between layers that are passed through unchanged and layers that are modified. Each coupling stage includes the computation of two functions: scaling function s(·) for scaling the inputs and translation function t(·) for translating the inputs. Permutations are performed at each coupling stage to ensure that each original input is modified at least several times while passing through the stack of coupling layers. Each affine transformation is a step towards modifying the original input distribution to another desired target distribution.

The normalizing flows model is denoted as p(x), where x is input data from a dataset. The normalizing flows model p(x) is a one-to-one mapping ƒ from x∈X to z∈Z. Normalizing flows model p(x) can be computed based on the change of variable formula as follows:

$${p}_{X} ( x ) = {p}_{Z} ( f ( x ) ) \vert det ( \frac{\partial f ( x )}{\partial {x}^{T}} ) \vert \qquad ( Equation 3 )$$

$$log ( {p}_{X} ( x ) ) = log ( {p}_{Z} ( f ( x ) ) ) + log ( \vert det ( \frac{\partial f ( x )}{\partial {x}^{T}} ) \vert ) \qquad ( Equation 4 )$$

The original input data x is projected through ƒ onto z∈Z, where z is a latent variable. In one example, p_{Z} may be a simple multivariate Gaussian distribution. The second term in Equation 4 describes the amount of space stretch or squeeze that is performed by the normalizing flows model p(x) around x. A loss function may be applied to maximize log(p_{X}(x)) for all x∈X. The underlying idea of this approach is to use a simple distribution (for which densities can be easily and quickly computed) to group the nonlinear embeddings of the original input data x. The second term in Equation 4 imposes the restriction that ƒ must be bijective.

Once trained, the trained medical analysis network and the trained audit network may be applied during an online or inference stage. For example, as shown in FIG. 2, the medical analysis network and the audit network are applied during online stage 204. In one example, the trained medical analysis network and the trained audit network are applied to respectively perform steps 104 and 106 of FIG. 1. The trained audit network can be used to flag input data of low probability, such as, e.g., input medical data that does not resemble (i.e., out-of-distribution with respect to) the training dataset on which the medical analysis network is trained. The medical analysis network performing the medical analysis task on such flagged input medical data can lack robustness, given that the flagged input data is outside of the training distribution.

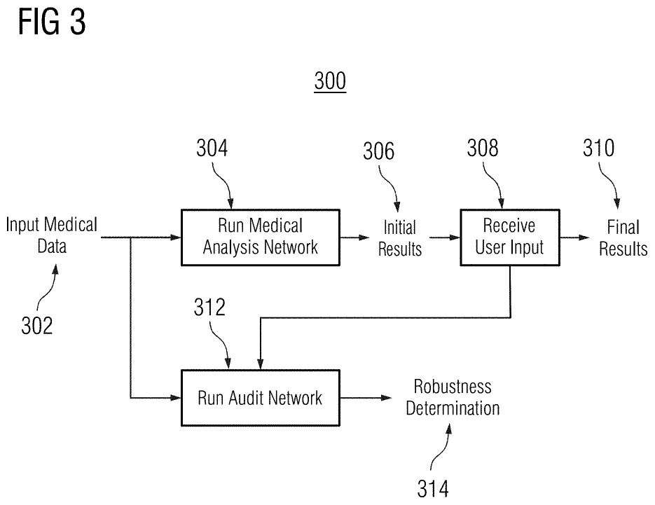

In one embodiment, the audit network may be applied for evaluating user input received for performing the medical analysis task. Certain medical analysis tasks are not fully automated and may involve user input, for example, to edit results of the medical analysis network. In such situations where the medical analysis task involves user input, the audit network may be applied to evaluate whether the user input is likely or unlikely to be correct, as shown in FIG. 3.

FIG. 3 shows a workflow 300 for evaluating user input received for performing a medical analysis task, in accordance with one or more embodiments. Workflow 300 shows an updated online stage of online stage 204 of FIG. 2. In workflow 300, a medical analysis network receives input medical data 302 and is run at block 304 for performing a medical analysis task to generate initial results 306. User input is received at block 308, e.g., for editing initial results 306 to generate final results 310. An audit network receives input medical data 302, initial results 306, and final results 310 as input and the audit network is run at block 312 to determine robustness determination 314. The audit network further evaluates whether final results 310, which incorporate the user input, is correct (i.e., can be trusted). The audit network may be trained (during a prior offline or training stage) on pairs of training medical data and corresponding final results.

One example of a medical analysis task involving user input is semi-automated segmentation. The medical analysis network outputs a proposed segmentation as initial results 306. User input may be received to correct the proposed segmentation as final results 310. The audit network may then evaluate whether the corrected proposed segmentation is correct. Where the input medical data is determined to be out-of-distribution, final results 310 may be correct but the robustness determination 314 output from the audit network may indicate that final results 310 cannot be trusted. In this case, the user may overrule the audit network and/or the input medical data may be utilized for retraining the medical analysis network and the audit network.

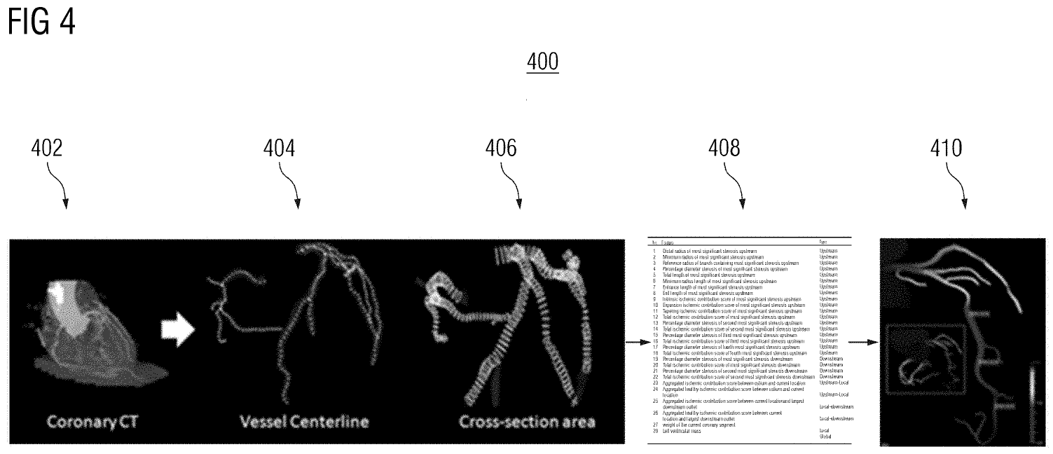

In one embodiment, a plurality of medical analysis networks and audit networks may be employed for the computation of FFR (fractional flow reserve). FIG. 4 shows a workflow 400 for calculating FFR from a CCTA (coronary CT angiography) image, in accordance with one or more embodiments. The computation of the FFR utilizes three medical analysis networks: a centerline detection network, a segmentation network, and an FFR computation network. The centerline detection network receives CCTA image 402 of vessels of a patient as input and generates vessel centerline image 404 identifying centerlines of the vessels as output. The segmentation network receives CCTA image 402 of the vessels and the vessel centerline image 404 as input and generates segmentation map 406 identifying a segmentation of the cross-section area along the centerlines of the vessels as output. The FFR computation network receives a set of features computed based on the centerlines in vessel centerline image 404 and the cross-sectional area in segmentation map 406 as input and generates calculated FFR values 408 for each location along the centerlines of the vessels. The centerline detection network and the segmentation network are followed by user input steps via user interface 410 where user input is received to correct/prune/add centerlines and correct the segmentation/cross-sectional area respectively. An audit network is trained for each medical analysis network. The audit networks may or may not take into consideration the user input steps when performing the evaluation. Three use cases related to the calculation of FFR are described below in accordance with one or more embodiments.

In a first use case, an audit network may be applied to detect artifacts in CCTA images along the coronary artery centerlines. In this use case, the input medical data comprises image patches of 32×32 pixels perpendicular to the centerlines, with spacing of 0.5 mm (millimeters) between patches. Each cross-section is labelled as follows: heathy, diseased, motion artifact, stent, ignore. The input to the audit network is either individual 2D cross-sections or 3D patches comprising multiple adjacent 2D cross-sections.

The same data preprocessing and data augmentation techniques may be applied for both the audit network and the medical analysis network. In general, the audit network and the medical analysis network share the same set of training data. Training of the audit network is performed end-to-end in a similar manner and time frame as the medical analysis network.

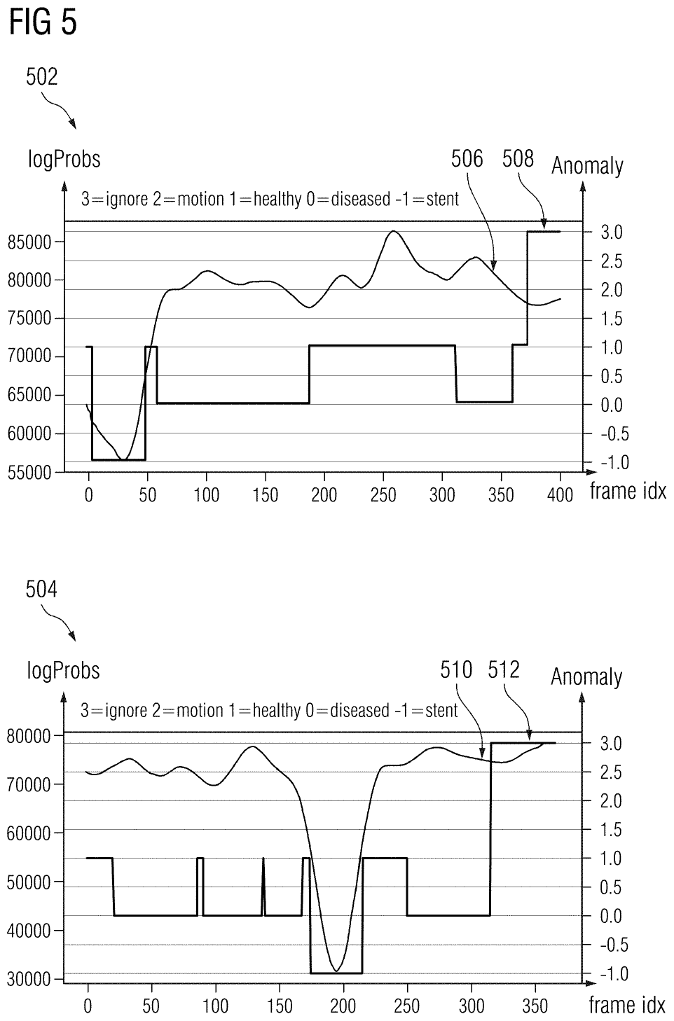

In an experimental evaluation, the audit network was trained on 3D patches of 16 adjacent cross-sections. The audit network was applied on an entire vessel using a sliding window approach. Only “healthy” cross-sections without artifacts were utilized for training, therefore making cross-sections with artifacts out-of-distribution with respect to the training data. FIG. 5 shows graphs 502 and 504 comparing robustness determined for independent input datasets in accordance with embodiments described herein with ground truth labels. Lines 506 and 510 represent a probability that input medical data is robust as determined by an audit network in accordance with embodiments described herein. Lines 508 and 512 represent ground truth labels where 3 indicates ignore, 2 indicates motion artifacts, 1 indicates healthy, 0 indicates diseased, and −1 indicates stent. As can be seen in graphs 502 and 504, the audit network outputs a significantly lower probability for the cross-sections of the stented regions. A correct detection of the stented segments is of particular interest since a reliable segmentation is difficult to perform in these regions.

In one embodiment, an additional machine learning based network (employing only dense layers) can be added on top of the z embedding provided by the audit network ƒ. This top-level network can be, for example, a classifier which detects the type of artifact that is present given a low probability cross-section. Since the embedded z vectors are constructed such that they are compared against a multivariate Gaussian distribution, which is a much simpler distribution compared to the distribution of pixels in the original image space, the new top-level classifier is able to reliably differentiate between artifact types.

In a second use case, the correctness of cross-sectional lumen contours is evaluated using an audit network. The cross-sectional lumen contours are obtained after automated segmentation (from a segmentation network) and manual editing. The input medical data comprises image patches of 32×32 pixels perpendicular to the centerline paired with the corresponding lumen contour. The input to the audit network can be a 4-dimensional tensor of sizes: 2 channels (the cross-section image and the lumen mask), 8 adjacent pairs of cross-sections and masks (yielding 4 mm of depth context), and a 2D resolution of 32×32 pixels.

In one embodiment, one or more artificial mask perturbations can be applied for increasing the audit network's sensitivity on certain types of mask defects. Exemplary artificial mask perturbations include: 1) zooming in on a region-of-interest around the lumen mask to make the audit network more aware of over- and under-segmentation; 2) translating the lumen mask to model potential offset between cross-sections and proposed segmentations; and 3) morphing the lumen mask along multiple directions (e.g., extruding or shrinking the lumen mask along a (e.g., random) axis) to model structural mask defects in which a portion of the mask is wrongfully including or excluding a small region of the cross-section while the rest of the mask is correct.

The training data for the audit network can be constructed from the same training data as used in the development of the medical analysis network. The loss function may be modified as follows. If the training data is untouched (i.e., as observed by the medical analysis network), the audit network's probability output is maximized. If the training data was perturbed, a hinge loss can be employed to force the audit network's probability output to be under a certain value, much lower than the probability value pertaining to the untouched training data.

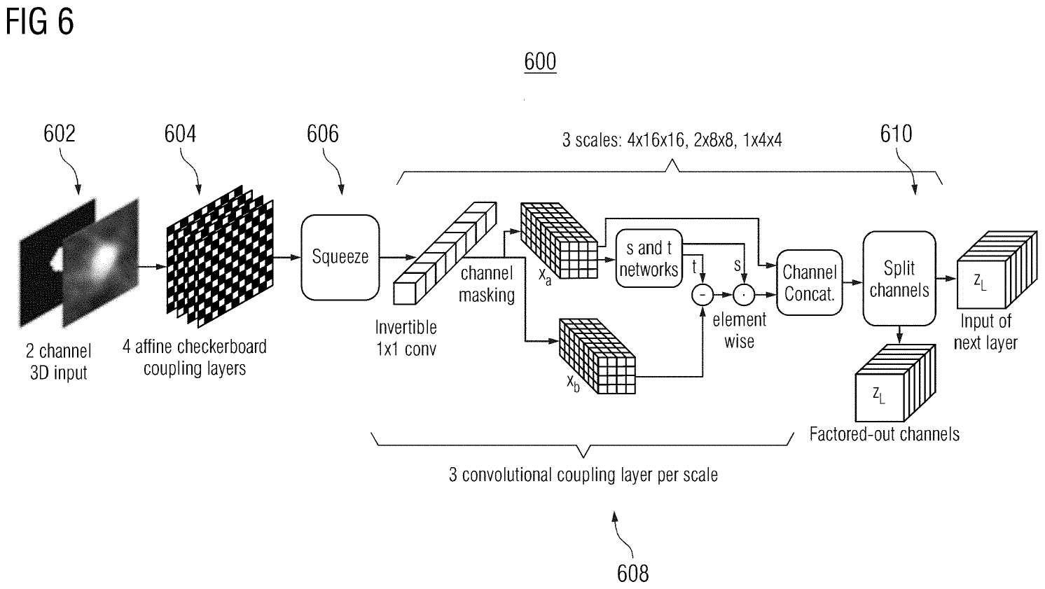

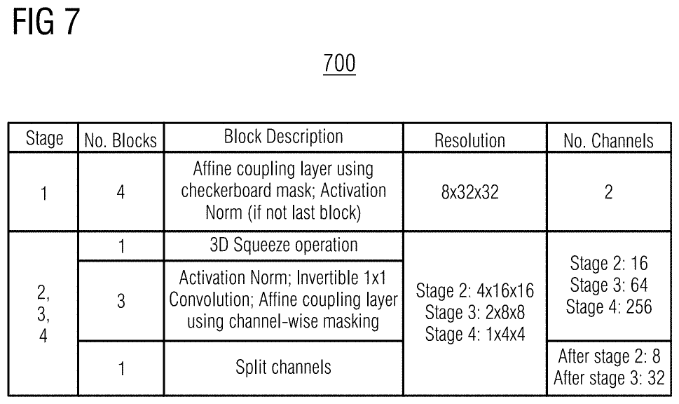

In one embodiment, the audit network may be implemented with a Glow-style normalizing flow architecture, combining layers such as, e.g., checkerboard and channel masking coupling layers, invertible 1×1 convolutions, split and squeeze layers, etc. FIG. 6 shows an exemplary Glow-style normalizing flows network architecture 600 for an audit network, in accordance with one or more embodiments. Glow-style normalizing flows network architecture 600 may be implemented according to table 700 of FIG. 7, in accordance with one or more embodiments.

Glow-style normalizing flows network architecture 600 comprises 4 stages, as described in Table 1. Stage 1 comprises 4 affine checkerboard coupling layers 604. Affine checkerboard coupling layers 604 receive input medical images 602 as input. Input medical images 602 comprises a 2 channel (the concatenation of the CTA and the binary mask volumes) 3D image having an 8×32×32 resolution (8 slices of 32×32 width/height). Three squeeze operations 606 contract the input resolution 2³ times down to 1×4×4, with increasing number of channels.

After each squeeze operation 606, 3 convolutional coupling layers 608 are applied for 3 scales: 4×16×16, 2×8×8, and 1×4×4. Coupling layers 608 apply the operations as defined by Equations 5 and 6. The effective receptive field of a coupling layer is given by the receptive field of the scaling function s and translation function t, in this case 5×5×5. Stacking coupling layers and using multiple scales (i.e., squeeze layers) increases the final normalizing flows receptive field, similar to the operation of classical CNNs.

yₐ=xₐ (Equation 5)

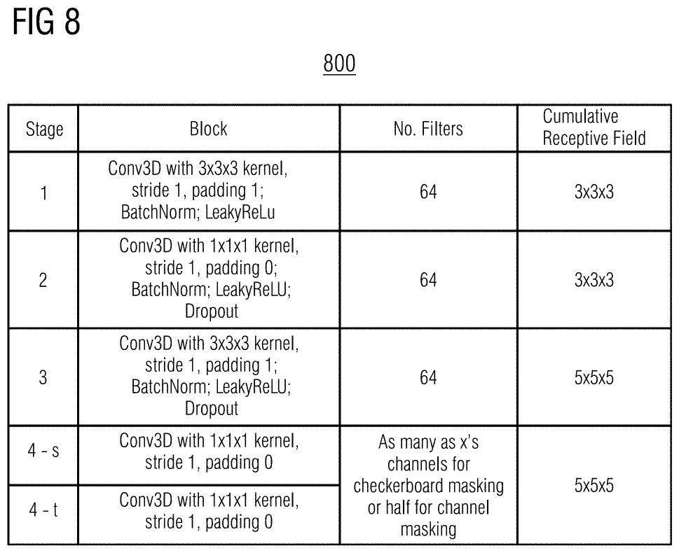

y_{b}=(x_{b}−t_{DNN}(xₐ))s_{DNN}(xₐ) (Equation 6)

where x and y are the input and output tensors respectively. Subscripts a and b denote two halves of the tensors: a first half which is passed-through unchanged and a remaining second half which is updated in a linear fashion with respect to itself, but in a highly non-linear fashion with respect to the first half through scaling function s and translation function t which are DNNs (deep neural networks). Functions s and t may be implemented as a two-headed 3D CNN (convolutional neural network) according to table 800 of FIG. 8, in accordance with one or more embodiments. In one example, the final activation function of s may be exp(tanh(x)) to easily compute the contribution to logDet (as Σ tanh(x) across all spatial dimensions and channels) and provide a bound of [e⁻¹, e¹] to the scalding performed at each coupling layer, ensuring numerical stability and a bounded global maximal value of logDet. Splitting layers 610 split the tensors, e.g., in half along the channel axis so that half of the channels are retained as input to the next layer of the computation graph while the remaining half of the channels are factored out.

In one embodiment, a coupling layer is provided that can operate efficiently for both normalizing flows directions, does not focus on local pixel correlations, and has an inductive bias similar to conventional CNNs. The coupling layer resembles a standard Glow-like sequence of 1×1 convolution (with applied bias) whose parameters are computed based on the passed-through channels. The applied bias is broadcasted to all spatial positions, and is therefore the same across the width, height, and depth of the resulting tensor, meaning that the coupling layer is no longer capable to reproduce masked pixel values. The same (sample specific) convolution kernel is applied at all spatial positions, in contrast to an element-wise computation. Equations 7 and 8 describe the coupling layer's operation as follows.

yₐ=xₐ (Equation 7)

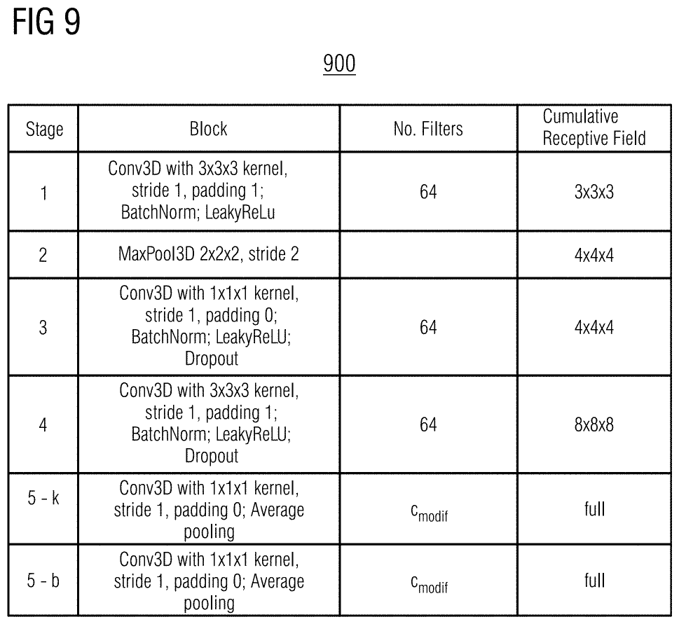

y_{b}=xₐ*k(xₐ)+b(xₐ) (Equation 8)

where * denotes a 1×1 convolution with kernel k and + denotes a broadcasting sum. k is computed by a CNN and has a shape c_{modif}×c_{modif}, where c_{modif} is the number of channels that are updated. b is a vector of c_{modif} elements. The CNN for computing k and b is implemented according to table 900 of FIG. 9, in accordance with one or more embodiments.

The coupling layer is self-conditioned (i.e., it does not employ an external conditioning network or another parallel flow) since the lumen binary mask and the angiographic image were not treated separately but were concatenated on the channel axis. This is possible because the mask and the image should be highly correlated spatially in order to achieve high log-probability.

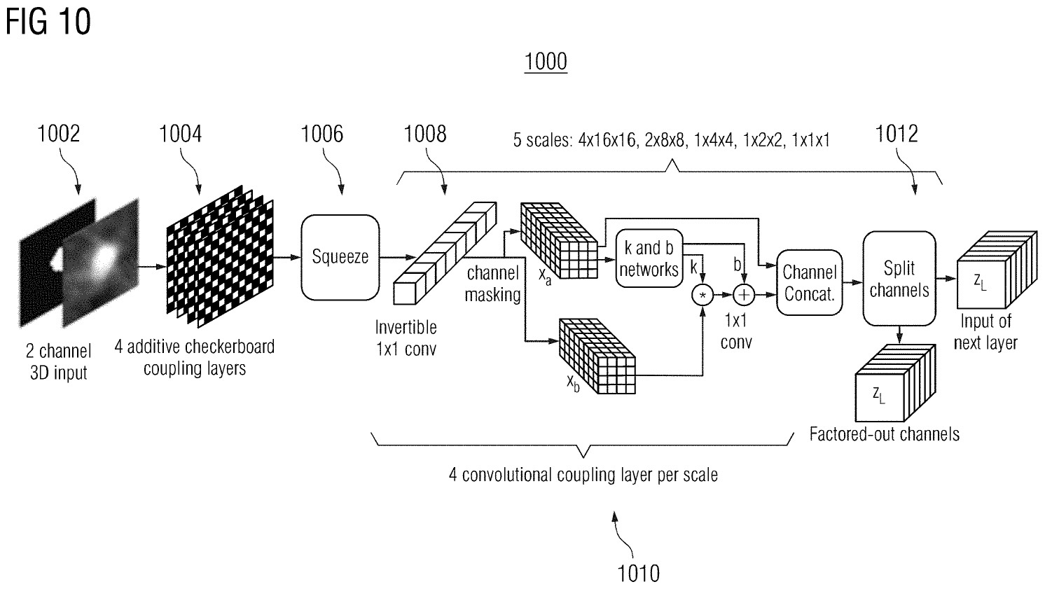

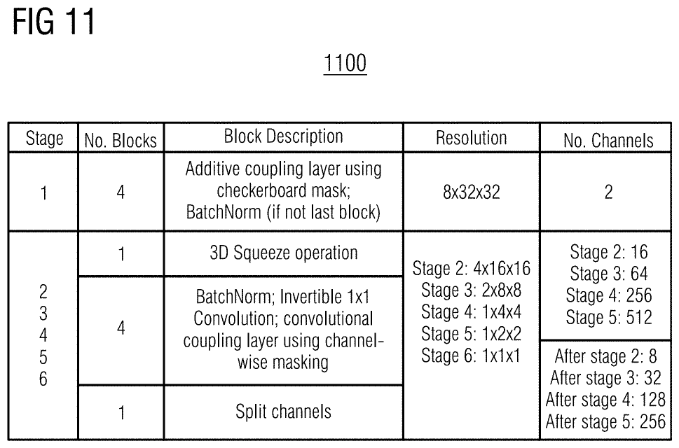

FIG. 10 shows a network architecture 1000 for implementing a normalizing flows audit network, in accordance with one or more embodiments. Network architecture 1000 employs the coupling layer defined by Equations 7 and 8. Network architecture 100 is implemented according to table 11 of FIG. 11, in accordance with one or more embodiments.

Stage 1 of network architecture 1000 comprises coupling layers 1004. Coupling layers 1004 receive input medical data 1002 as input. Coupling layers 1004 comprises a sequence of additive coupling layers with checkerboard masking. Coupling layers 1004 focus mainly on local pixel correlations. As opposed to affine couplings, additive couplings are volume preserving (i.e., they do not contribute directly to logDet and the final log(p(x)), but indirectly through the upstream layers).

Stage 2 of network architecture 1000 comprises cascades of coupling layers. In contrast to a classical CNN where filters of shape 3×3 (or larger) and strides larger than 1 are used (either in convolutional or max pool layers) to increase the effective FOV (field-of-view), the FOV of network architecture 1000 is increased solely by squeeze operations 1006. After squeeze operations 1006, a 1×1×1 patch of pixels is formed from a patch of 2×2×2 pixels, which were flattened spatially into the channel dimension. Therefore, the FOV doubles on each spatial axis for each squeeze step. This allows invertible 1×1 convolutions 1008 to operate on increasingly larger FOVs, while still retaining the capability of efficient forward/backward normalizing flows computation. There are enough squeeze operations 1006 so that the resolution on the last stage decays to 1×1×1. The input spatial dimensions are restricted to be powers of 2.

After each squeeze operation 1006, 4 convolutional coupling layers 1010 are applied for 5 scales: 4×16×16, 2×8×8, 1×4×4, 1×2×2, and 1×1×1. Coupling layers 1010 apply the operations as defined by Equations 7 and 8. The number of channels cᵢ (at stage i) increases exponentially with the number of squeezed dimensions, as shown in table 1100 of FIG. 11. This directly impacts the coupling layer's runtime and complexity since it must produce matrix k whose size scales with the square of cᵢ. Also, inference and sampling involve computing the determinant and inverse of k respectively. To alleviate this issue, the splitting layers 1012 may be modified so that the tensors are not split in half along the channel axis but instead only, e.g., a quarter is retained as input to the next layer of the computation graph while the remaining 75% of the channels are factored out. This can be applied in stage 1, where the embeddings mostly describe texture. After such a split, the input to the next squeeze has only cᵢ/4 channels, half of those in a regular split. Cascading such split throughout the network can alleviate the effect of the exponentially-increasing cᵢ, particularly for larger resolution inputs. In one embodiment, the first split layer may only retain cᵢ/4 channels. The net effect is that there are only 512 channels in the last stage, as opposed to the original 1024 (as shown in table 11 of FIG. 11), resulting in faster runtimes and fewer model parameters.

In network architecture 1000, BatchNorm is employed instead of ActNorm. Two running averages of the batch mean, and standard-deviation are employed for normalization and are updated with current batch statistics after their use, so that the normalization procedure is dependent only on past batches and any cross-talk between samples in the current batch is eliminated. BatchNorm's main purpose is to provide “checkpoints” for activations inside the network (i.e., after each BatchNorm layer, the activations have preset statistics (e.g., are centered around 0 with a standard deviation of 1)) to improve the training process.

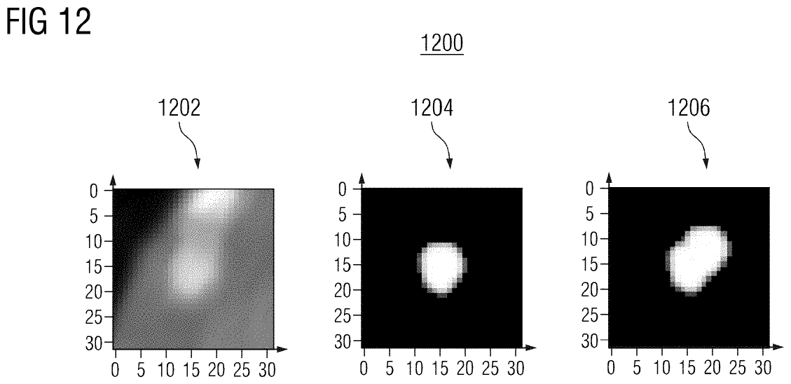

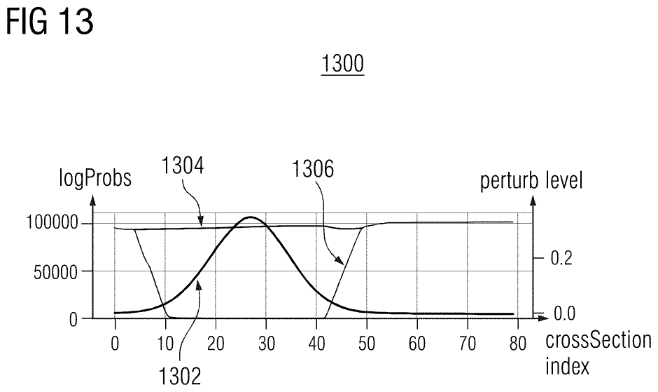

A normalizing flows audit network implemented according to network architecture 1000 of FIG. 10 was experimentally validated. FIG. 12 shows training images 1200 used for training the normalizing flows audit network, in accordance with one or more embodiments. Image 1202 shows an original cross-section image of a vessel and image 1204 shows a corresponding lumen mask. Image 1206 shows a perturbed lumen mask by applying a mask extrusion perturbation of 20% severity on the bottom-left to top-right direct of image 1204. FIG. 13 shows a graph 1300 showing the probability variation across one vessel segment of 80 cross-sections (equivalent to 40 mm depth) from a test dataset, in accordance with one or more embodiments. A mask perturbation is applied at a random location and with randomly chosen duration across cross-sections. The severity of the perturbation is denoted by line 1302 (as defined by the right y-axis). The normalizing flows audit network's output (log-probability) prior to applying the perturbations) is denoted by line 1304 (as defined by the left y-axis). The normalizing flows audit network's output (log-probability) after applying the perturbations is denoted by line 1306 (as defined by the left y-axis). The normalizing flows audit network detects the presence of the mask perturbation and, as a result, its probability output dips when the perturbation level is large enough.

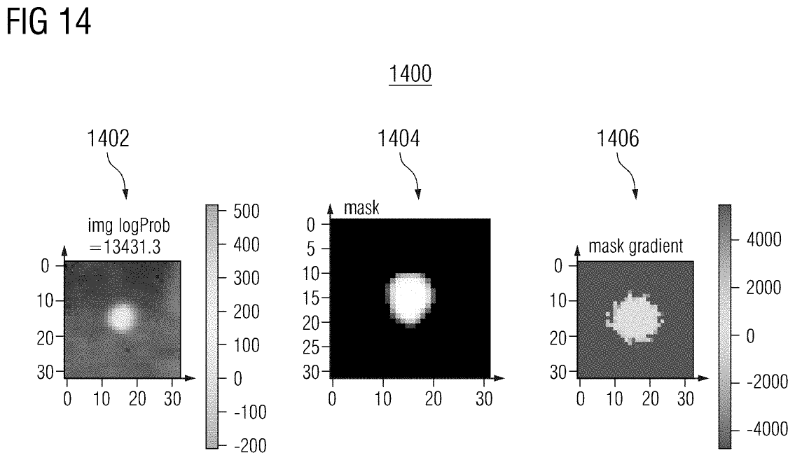

FIG. 14 shows saliency plots 1400 for a normalizing flows audit model, in accordance with one or more embodiments. Plot 1402 shows an image gradient over-imposed on a cross-section image. Plot 1404 shows the original lumen mask. Plot 1406 shows the lumen mask pixel-wise gradient. The scale of mask gradient in plot 1406 is larger in magnitude than the scale of the cross-section image gradient in plot 1402. As shown in plot 1404, the gradient inside the mask is almost zero while the gradient on the edges and outside are larger (i.e., the log-prob would increase if some edge pixels were to increase their intensity but would immediately decay if any pixel outside the mask neighborhood was set to 1). Saliency plots 1400 reveal that the normalizing flows audit model focuses both on the content of the cross-sectional image and on the provided lumen mask but penalizes more severely the boundary of the lumen mask. Thus, small deviations on the mask boundary lead to large reductions in the predicted log-probability, suggesting that the normalizing flows audit model trained using synthetic mask perturbations checks if the mask is correctly aligned with the cross-section.

In a third use case, an evaluation of whether the FFR can be reliably computed by determining whether the feature vector for a given centerline location for computing the FFR value lies within the distribution of the training data on which the FFR computation network is trained. The FFR computation network may be trained based on synthetic data and evaluated on synthetic and real patient data. The audit network is implemented as a normalizing flows model to estimate the probability density for input medical data to determine how likely the input medical data is to be similar (e.g., in the same distribution) to the synthetic data on which the FFR computation network is trained.

For this use case, the same training dataset may be used to develop both the FFR computation network and the audit network. In one experimental implementation, the audit network was a normalizing flows architecture, which employed stacks of coupling layers. The audit network was found to be fast and lightweight since it operated on 0D data.

The synthetic training data was split at case level: 90% was used as the training dataset and the remaining 10% was used as the validation set for the normalizing flows audit network. A patient dataset was employed as test-set only. The normalizing flows model for implementing the audit network was selected such that the log-probability of its training dataset is close to the log-probability of its validation set and the separation between log-probabilities of random features and the log-probabilities of real data features is maximized. The probabilities obtained using the audit network were also aggregated at the patient level by averaging over all centerline locations.

To evaluate the performance of the audit network, another experiment was conducted. For a subset of features, for each feature, the sample with the highest value of that feature was determined (add 1-10 standard deviations) and the sample with the lowest value of that feature was determined (subtract 1-10 standard deviations). It was found that the log probabilities decrease gradually as the values of the features become more unlikely.

In another experiment, the value of the feature “percent diameter stenosis of the main stenosis upstream” was modified in increments/decrements of 5%. It was found that the log-probabilities decrease once the values change more than +/−10% (even though the absolute value is still a probable one). Thus, the audit network learns the relationship between features and detects abnormal feature value combinations.

In one embodiment, embodiments described herein may be applied to increase the success rate of a clinical center by reducing the number of rejected cases in the clinical center. Depending on the equipment employed for data acquisition and on the experience of the clinicians, the number of cases rejected by the audit network may vary. It is in the best interest of both the clinical center and the manufacturer of the equipment/developer of the medical analysis network to minimize the number of rejected cases using the audit network. While the presence of the out-of-distribution input medical data rejected by the audit network cannot be controlled or minimized directly (this can be addressed in an indirect centralized manner by collecting as many out-of-distribution cases as possible and iteratively improving the medical analysis network and the audit network), the number of cases with artifacts can be minimized. Cases of rejected cases due to faulty data acquisition and faulty user input are distinguished between.

For faulty data acquisition, cases rejected by the audit network are sent back to the manufacturer and suggestions for improving the data acquisition process are sent back to the clinical center. These suggestions may be related to, for example, the data acquisition protocol, equipment settings, equipment issues (e.g., maintenance or replacement of certain equipment components), etc. The suggestions may be determined automatically (e.g., using a machine learning based model based on natural language processing), semi-automatically, or manually.

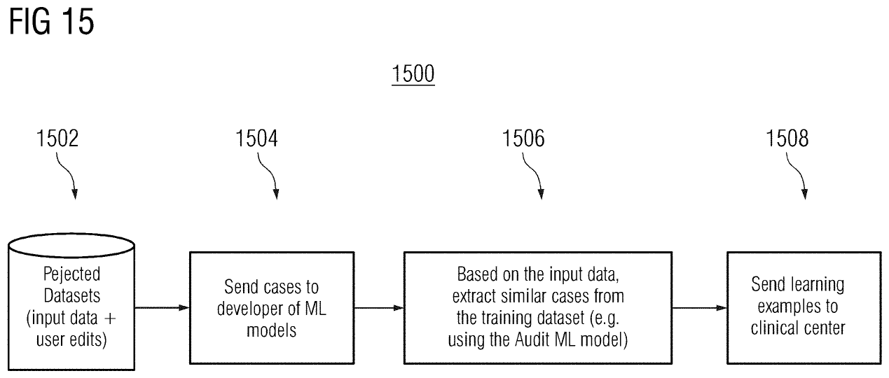

For faulty user input, cases rejected by the audit network may be due to incorrect edits or other user input by the user. One example of faulty user input may be with respect to segmentation of cross-sectional lumen contours, as described above. To reduce the number of rejected cases, clinicians should be trained to provide correct edits/inputs. Clinician training may be performed in a live session by experienced clinicians or may be performed automatically, for example, as described in FIG. 15. FIG. 15 shows a workflow 1500 for reducing the number of rejected cases in a clinical center, in accordance with one or more embodiments. Rejected datasets (including input data and user edits) due to faulty user input are stored in database 1502. At step 1504, the rejected cases are sent back to the developer of the machine learning models (e.g., the medical analysis network and the audit network). At step 1506, based on the rejected cases, similar cases are extracted from a training dataset. At step 1508, the extracted input/output pairs are sent to the clinical center as learning examples for training clinicians. Where the audit network is implemented as a normalizing flows audit network, the normalizing flows audit network may be utilized to extract the similar cases. The audit network's predicted log-probability value depends both on the z embeddings of input medical data x and on the amount of space stretch/squeeze performed by the normalizing flows model (i.e., the second term in Equation 4). Given a query sample x_{q}, the closest samples from the dataset D can be found by evaluating a heuristic based on vector distance (between dataset embeddings z_{D} and the query sample's embedding z_{q}) and the amount of space stretch/squeeze around each sample x∈D and x_{q}. Given a multi-scale normalizing flows architecture, the heuristic can also operate either only on top-level embedding components to find samples close to x_{q} or only on bottom-level embedding components to find samples to close x_{q} texture-wise.

In one embodiment, the outputs of the audit network may be utilized for assisted editing tasks. For example, in the case of image segmentation, in response to determining that the medical analysis network is not robust for performing the image segmentation, one or more alternate segmentations may be proposed to the user from other medical imaging analysis networks (e.g., as resulting from ensemble machine learning based models), which give higher scores of agreement with the original training dataset. The user may directly edit the proposed segmentations or an interaction mechanism (e.g., an on-screen slider) can be provided which allows continuous representations along a direction of increasing/decreasing scores of confidence. Each of the proposed segmentations can be presented with the output to the audit network to thus allow the user to choose an option which is acceptable according to the audit network.

In one embodiment, the output of the audit network may be used to automatically correct the output of the medical analysis network. For example, in image segmentation, the predicted segmentation mask can be optimized with respect to the output of the audit network to increase the similarity score with the original training dataset. In one embodiment, an iterative procedure may be employed in which the audit network is viewed as a function to be maximized through its input (i.e., the segmentation mask at the current iteration). In another embodiment, a saliency map of the input segmentation mask may be computed and heuristics may be utilized to obtain a segmentation mask with a higher similarity score. This approach has the advantage that it may be performed in a single step as opposed to an iterative procedure.

In one embodiment, user input (e.g., editing or other interaction by the user) received during the online stage may be used to learn updates to both the medical analysis network and the audit network.

In one embodiment, the outputs from the medical analysis network and the audit network can be used in conjunction to identify additional high value datasets that would provide the most value from being part of the training dataset. In one example, when provided with a large new dataset comprising multiple samples, both the medical analysis network and the audit network may be run on the new dataset and the samples are sorted by order of decreasing scores of dissimilarities from the original training datasets. The datasets with the highest scores of dissimilarity can be annotated by a user and included in the training dataset for training an updated model. This approach can also be used during the online live utilization of the medical analysis network and audit network, where cases with high scores of dissimilarity requiring significant editing, can be flagged by the audit network and transferred for retraining the medical analysis network and audit network after suitable data clearing processes.

In one embodiment, where datasets show high scores of agreement, downstream processing tasks which depend on the outputs of the medical analysis network can be triggered in advance to obtain results faster, reducing total wait time for the user. Where no editing is required, the results can be shown to the user instantaneously. Where editing is needed, the results are updated.

In one embodiment, the output of the audit network may be used to infer an uncertainty for the medical analysis network. The uncertainty may be further used as input for clinical decision making (which in turn may be performed by a clinical or input into a higher order clinical decision support system).

Embodiments described herein are described with respect to the claimed systems as well as with respect to the claimed methods. Features, advantages or alternative embodiments herein can be assigned to the other claimed objects and vice versa. In other words, claims for the systems can be improved with features described or claimed in the context of the methods. In this case, the functional features of the method are embodied by objective units of the providing system.

Furthermore, certain embodiments described herein are described with respect to methods and systems utilizing trained machine learning based networks (or models), as well as with respect to methods and systems for training machine learning based networks. Features, advantages or alternative embodiments herein can be assigned to the other claimed objects and vice versa. In other words, claims for methods and systems for training a machine learning based network can be improved with features described or claimed in context of the methods and systems for utilizing a trained machine learning based network, and vice versa.

In particular, the trained machine learning based networks applied in embodiments described herein can be adapted by the methods and systems for training the machine learning based networks. Furthermore, the input data of the trained machine learning based network can comprise advantageous features and embodiments of the training input data, and vice versa. Furthermore, the output data of the trained machine learning based network can comprise advantageous features and embodiments of the output training data, and vice versa.

In general, a trained machine learning based network mimics cognitive functions that humans associate with other human minds. In particular, by training based on training data, the trained machine learning based network is able to adapt to new circumstances and to detect and extrapolate patterns.

In general, parameters of a machine learning based network can be adapted by means of training. In particular, supervised training, semi-supervised training, unsupervised training, reinforcement learning and/or active learning can be used. Furthermore, representation learning (an alternative term is “feature learning”) can be used. In particular, the parameters of the trained machine learning based network can be adapted iteratively by several steps of training.

In particular, a trained machine learning based network can comprise a neural network, a support vector machine, a decision tree, and/or a Bayesian network, and/or the trained machine learning based network can be based on k-means clustering, Q-learning, genetic algorithms, and/or association rules. In particular, a neural network can be a deep neural network, a convolutional neural network, or a convolutional deep neural network. Furthermore, a neural network can be an adversarial network, a deep adversarial network and/or a generative adversarial network.

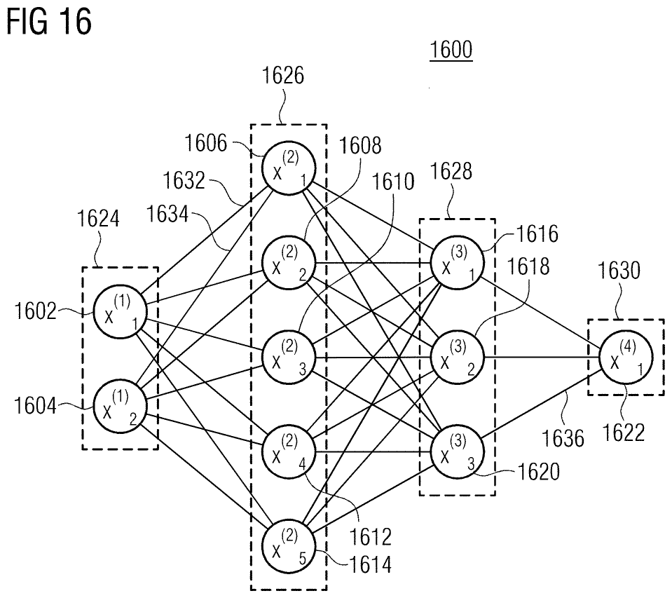

FIG. 16 shows an embodiment of an artificial neural network 1600, in accordance with one or more embodiments. Alternative terms for “artificial neural network” are “neural network”, “artificial neural net” or “neural net”. Machine learning networks described herein, such as, e.g., the machine learning based medical analysis network 104 and the machine learning based audit network 106 of FIG. 1 and the medical analysis network and the audit network of FIG. 2 and FIG. 3, may be implemented using artificial neural network 1600.

The artificial neural network 1600 comprises nodes 1602-1622 and edges 1632, 1634, . . . , 1636, wherein each edge 1632, 1634, . . . , 1636 is a directed connection from a first node 1602-1622 to a second node 1602-1622. In general, the first node 1602-1622 and the second node 1602-1622 are different nodes 1602-1622, it is also possible that the first node 1602-1622 and the second node 1602-1622 are identical. For example, in FIG. 16, the edge 1632 is a directed connection from the node 1602 to the node 1606, and the edge 1634 is a directed connection from the node 1604 to the node 1606. An edge 1632, 1634, . . . , 1636 from a first node 1602-1622 to a second node 1602-1622 is also denoted as “ingoing edge” for the second node 1602-1622 and as “outgoing edge” for the first node 1602-1622.

In this embodiment, the nodes 1602-1622 of the artificial neural network 1600 can be arranged in layers 1624-1630, wherein the layers can comprise an intrinsic order introduced by the edges 1632, 1634, . . . , 1636 between the nodes 1602-1622. In particular, edges 1632, 1634, . . . , 1636 can exist only between neighboring layers of nodes. In the embodiment shown in FIG. 16, there is an input layer 1624 comprising only nodes 1602 and 1604 without an incoming edge, an output layer 1630 comprising only node 1622 without outgoing edges, and hidden layers 1626, 1628 in-between the input layer 1624 and the output layer 1630. In general, the number of hidden layers 1626, 1628 can be chosen arbitrarily. The number of nodes 1602 and 1604 within the input layer 1624 usually relates to the number of input values of the neural network 1600, and the number of nodes 1622 within the output layer 1630 usually relates to the number of output values of the neural network 1600.

In particular, a (real) number can be assigned as a value to every node 1602-1622 of the neural network 1600. Here, x⁽ⁿ⁾ᵢ denotes the value of the i-th node 1602-1622 of the n-th layer 1624-1630. The values of the nodes 1602-1622 of the input layer 1624 are equivalent to the input values of the neural network 1600, the value of the node 1622 of the output layer 1630 is equivalent to the output value of the neural network 1600. Furthermore, each edge 1632, 1634, . . . , 1636 can comprise a weight being a real number, in particular, the weight is a real number within the interval [−1, 1] or within the interval [0, 1]. Here, w^{(m,n)}_{i,j} denotes the weight of the edge between the i-th node 1602-1622 of the m-th layer 1624-1630 and the j-th node 1602-1622 of the n-th layer 1624-1630. Furthermore, the abbreviation w⁽ⁿ⁾_{i,j} is defined for the weight w^{(n,n+1)}_{i,j}.

In particular, to calculate the output values of the neural network 1600, the input values are propagated through the neural network. In particular, the values of the nodes 1602-1622 of the (n+1)-th layer 1624-1630 can be calculated based on the values of the nodes 1602-1622 of the n-th layer 1624-1630 by

xⱼ⁽ⁿ⁺¹⁾=f(Σᵢxᵢ⁽ⁿ⁾·w_{i,j}⁽ⁿ⁾).

Herein, the function f is a transfer function (another term is “activation function”). Known transfer functions are step functions, sigmoid function (e.g. the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 1624 are given by the input of the neural network 1600, wherein values of the first hidden layer 1626 can be calculated based on the values of the input layer 1624 of the neural network, wherein values of the second hidden layer 1628 can be calculated based in the values of the first hidden layer 1626, etc.

In order to set the values w^{(m,n)}_{i,j} for the edges, the neural network 1600 has to be trained using training data. In particular, training data comprises training input data and training output data (denoted as tᵢ). For a training step, the neural network 1600 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 1600 (backpropagation algorithm). In particular, the weights are changed according to

w_{i,j}^{τ(n)}=w_{i,j}⁽ⁿ⁾−γ·δⱼ⁽ⁿ⁾·xᵢ⁽ⁿ⁾

wherein γ is a learning rate, and the numbers δ⁽ⁿ⁾ⱼ can be recursively calculated as

δⱼ⁽ⁿ⁾=(Σₖδₖ⁽ⁿ⁺¹⁾·w_{j,k}⁽ⁿ⁺¹⁾)·f^{τ}(Σᵢxᵢ⁽ⁿ⁾·w_{i,j}⁽ⁿ⁾)

based on δ⁽ⁿ⁺¹⁾ⱼ, if the (n+1)-th layer is not the output layer, and

δⱼ⁽ⁿ⁾=(xₖ⁽ⁿ⁺¹⁾−tⱼ⁽ⁿ⁺¹⁾)·f^{τ}(Σᵢxᵢ⁽ⁿ⁾·w_{i,j}⁽ⁿ⁾)

if the (n+1)-th layer is the output layer 1630, wherein f′ is the first derivative of the activation function, and y⁽ⁿ⁺¹⁾ⱼ is the comparison training value for the j-th node of the output layer 1630.

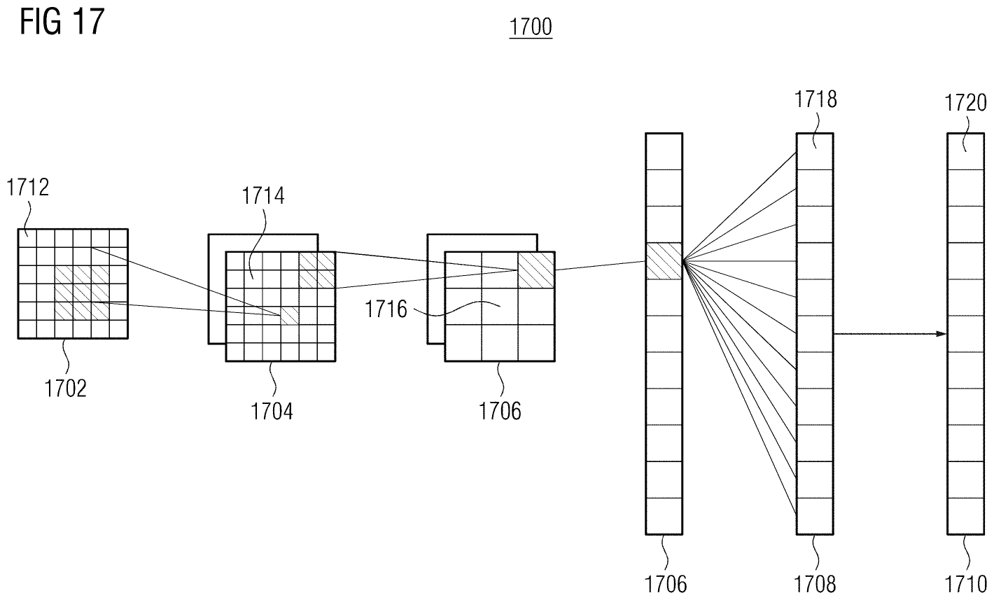

FIG. 17 shows a convolutional neural network 1700, in accordance with one or more embodiments. Machine learning networks described herein, such as, e.g., the machine learning based medical analysis network 104 and the machine learning based audit network 106 of FIG. 1 and the medical analysis network and the audit network of FIG. 2 and FIG. 3, may be implemented using convolutional neural network 1700.

In the embodiment shown in FIG. 17, the convolutional neural network comprises 1700 an input layer 1702, a convolutional layer 1704, a pooling layer 1706, a fully connected layer 1708, and an output layer 1710. Alternatively, the convolutional neural network 1700 can comprise several convolutional layers 1704, several pooling layers 1706, and several fully connected layers 1708, as well as other types of layers. The order of the layers can be chosen arbitrarily, usually fully connected layers 1708 are used as the last layers before the output layer 1710.

In particular, within a convolutional neural network 1700, the nodes 1712-1720 of one layer 1702-1710 can be considered to be arranged as a d-dimensional matrix or as a d-dimensional image. In particular, in the two-dimensional case the value of the node 1712-1720 indexed with i and j in the n-th layer 1702-1710 can be denoted as x⁽ⁿ_{)[i,j]}. However, the arrangement of the nodes 1712-1720 of one layer 1702-1710 does not have an effect on the calculations executed within the convolutional neural network 1700 as such, since these are given solely by the structure and the weights of the edges.

In particular, a convolutional layer 1704 is characterized by the structure and the weights of the incoming edges forming a convolution operation based on a certain number of kernels. In particular, the structure and the weights of the incoming edges are chosen such that the values x⁽ⁿ⁾ₖ of the nodes 1714 of the convolutional layer 1704 are calculated as a convolution x⁽ⁿ⁾ₖ=Kₖ*x⁽ⁿ⁻¹⁾ based on the values x⁽ⁿ⁻¹⁾ of the nodes 1712 of the preceding layer 1702, where the convolution * is defined in the two-dimensional case as

xₖ⁽ⁿ⁾[i,j]=(Kₖ*x⁽ⁿ⁻¹⁾)[i,j]=Σᵢ¹Σⱼ¹Kₖ[i¹,j¹]·x⁽ⁿ⁻¹⁾[i−i¹, j−j¹].

Here the k-th kernel Kₖ is a d-dimensional matrix (in this embodiment a two-dimensional matrix), which is usually small compared to the number of nodes 1712-1718 (e.g. a 3×3 matrix, or a 5×5 matrix). In particular, this implies that the weights of the incoming edges are not independent, but chosen such that they produce said convolution equation. In particular, for a kernel being a 3×3 matrix, there are only 9 independent weights (each entry of the kernel matrix corresponding to one independent weight), irrespectively of the number of nodes 1712-1720 in the respective layer 1702-1710. In particular, for a convolutional layer 1704, the number of nodes 1714 in the convolutional layer is equivalent to the number of nodes 1712 in the preceding layer 1702 multiplied with the number of kernels.

If the nodes 1712 of the preceding layer 1702 are arranged as a d-dimensional matrix, using a plurality of kernels can be interpreted as adding a further dimension (denoted as “depth” dimension), so that the nodes 1714 of the convolutional layer 1704 are arranged as a (d+1)-dimensional matrix. If the nodes 1712 of the preceding layer 1702 are already arranged as a (d+1)-dimensional matrix comprising a depth dimension, using a plurality of kernels can be interpreted as expanding along the depth dimension, so that the nodes 1714 of the convolutional layer 1704 are arranged also as a (d+1)-dimensional matrix, wherein the size of the (d+1)-dimensional matrix with respect to the depth dimension is by a factor of the number of kernels larger than in the preceding layer 1702.

The advantage of using convolutional layers 1704 is that spatially local correlation of the input data can exploited by enforcing a local connectivity pattern between nodes of adjacent layers, in particular by each node being connected to only a small region of the nodes of the preceding layer.

In embodiment shown in FIG. 17, the input layer 1702 comprises 36 nodes 1712, arranged as a two-dimensional 6×6 matrix. The convolutional layer 1704 comprises 72 nodes 1714, arranged as two two-dimensional 6×6 matrices, each of the two matrices being the result of a convolution of the values of the input layer with a kernel. Equivalently, the nodes 1714 of the convolutional layer 1704 can be interpreted as arranges as a three-dimensional 6×6×2 matrix, wherein the last dimension is the depth dimension.

A pooling layer 1706 can be characterized by the structure and the weights of the incoming edges and the activation function of its nodes 1716 forming a pooling operation based on a non-linear pooling function f. For example, in the two dimensional case the values x⁽ⁿ⁾ of the nodes 1716 of the pooling layer 1706 can be calculated based on the values x⁽ⁿ⁻¹⁾ of the nodes 1714 of the preceding layer 1704 as

x⁽ⁿ⁾[i,j]=f(x⁽ⁿ⁻¹⁾[id₁,jd₂], . . . , x⁽ⁿ⁻¹⁾[id₁+d₁−1,jd₂+d₂−1])

In other words, by using a pooling layer 1706, the number of nodes 1714, 1716 can be reduced, by replacing a number d1·d2 of neighboring nodes 1714 in the preceding layer 1704 with a single node 1716 being calculated as a function of the values of said number of neighboring nodes in the pooling layer. In particular, the pooling function f can be the max-function, the average or the L2-Norm. In particular, for a pooling layer 1706 the weights of the incoming edges are fixed and are not modified by training.

The advantage of using a pooling layer 1706 is that the number of nodes 1714, 1716 and the number of parameters is reduced. This leads to the amount of computation in the network being reduced and to a control of overfitting.

In the embodiment shown in FIG. 17, the pooling layer 1706 is a max-pooling, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

A fully-connected layer 1708 can be characterized by the fact that a majority, in particular, all edges between nodes 1716 of the previous layer 1706 and the nodes 1718 of the fully-connected layer 1708 are present, and wherein the weight of each of the edges can be adjusted individually.

In this embodiment, the nodes 1716 of the preceding layer 1706 of the fully-connected layer 1708 are displayed both as two-dimensional matrices, and additionally as non-related nodes (indicated as a line of nodes, wherein the number of nodes was reduced for a better presentability). In this embodiment, the number of nodes 1718 in the fully connected layer 1708 is equal to the number of nodes 1716 in the preceding layer 1706. Alternatively, the number of nodes 1716, 1718 can differ.

Furthermore, in this embodiment, the values of the nodes 1720 of the output layer 1710 are determined by applying the Softmax function onto the values of the nodes 1718 of the preceding layer 1708. By applying the Softmax function, the sum the values of all nodes 1720 of the output layer 1710 is 1, and all values of all nodes 1720 of the output layer are real numbers between 0 and 1.

A convolutional neural network 1700 can also comprise a ReLU (rectified linear units) layer or activation layers with non-linear transfer functions. In particular, the number of nodes and the structure of the nodes contained in a ReLU layer is equivalent to the number of nodes and the structure of the nodes contained in the preceding layer. In particular, the value of each node in the ReLU layer is calculated by applying a rectifying function to the value of the corresponding node of the preceding layer.

The input and output of different convolutional neural network blocks can be wired using summation (residual/dense neural networks), element-wise multiplication (attention) or other differentiable operators. Therefore, the convolutional neural network architecture can be nested rather than being sequential if the whole pipeline is differentiable.

In particular, convolutional neural networks 1700 can be trained based on the backpropagation algorithm. For preventing overfitting, methods of regularization can be used, e.g. dropout of nodes 1712-1720, stochastic pooling, use of artificial data, weight decay based on the L1 or the L2 norm, or max norm constraints. Different loss functions can be combined for training the same neural network to reflect the joint training objectives. A subset of the neural network parameters can be excluded from optimization to retain the weights pretrained on another datasets.

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatus, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatus, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIGS. 1-3. Certain steps or functions of the methods and workflows described herein, including one or more of the steps or functions of FIGS. 1-3, may be performed by a server or by another processor in a network-based cloud-computing system. Certain steps or functions of the methods and workflows described herein, including one or more of the steps of FIGS. 1-3, may be performed by a client computer in a network-based cloud computing system. The steps or functions of the methods and workflows described herein, including one or more of the steps of FIGS. 1-3, may be performed by a server and/or by a client computer in a network-based cloud computing system, in any combination.

Systems, apparatus, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIGS. 1-3, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 1802 that may be used to implement systems, apparatus, and methods described herein is depicted in FIG. 18. Computer 1802 includes a processor 1804 operatively coupled to a data storage device 1812 and a memory 1810. Processor 1804 controls the overall operation of computer 1802 by executing computer program instructions that define such operations. The computer program instructions may be stored in data storage device 1812, or other computer readable medium, and loaded into memory 1810 when execution of the computer program instructions is desired. Thus, the method and workflow steps or functions of FIGS. 1-3 can be defined by the computer program instructions stored in memory 1810 and/or data storage device 1812 and controlled by processor 1804 executing the computer program instructions. For example, the computer program instructions can be implemented as computer executable code programmed by one skilled in the art to perform the method and workflow steps or functions of FIGS. 1-3. Accordingly, by executing the computer program instructions, the processor 1804 executes the method and workflow steps or functions of FIGS. 1-3. Computer 1802 may also include one or more network interfaces 1806 for communicating with other devices via a network. Computer 1802 may also include one or more input/output devices 1808 that enable user interaction with computer 1802 (e.g., display, keyboard, mouse, speakers, buttons, etc.).

Processor 1804 may include both general and special purpose microprocessors, and may be the sole processor or one of multiple processors of computer 1802. Processor 1804 may include one or more central processing units (CPUs), for example. Processor 1804, data storage device 1812, and/or memory 1810 may include, be supplemented by, or incorporated in, one or more application-specific integrated circuits (ASICs) and/or one or more field programmable gate arrays (FPGAs).

Data storage device 1812 and memory 1810 each include a tangible non-transitory computer readable storage medium. Data storage device 1812, and memory 1810, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 1808 may include peripherals, such as a printer, scanner, display screen, etc. For example, input/output devices 1808 may include a display device such as a cathode ray tube (CRT) or liquid crystal display (LCD) monitor for displaying information to the user, a keyboard, and a pointing device such as a mouse or a trackball by which the user can provide input to computer 1802.

An image acquisition device 1814 can be connected to the computer 1802 to input image data (e.g., medical images) to the computer 1802. It is possible to implement the image acquisition device 1814 and the computer 1802 as one device. It is also possible that the image acquisition device 1814 and the computer 1802 communicate wirelessly through a network. In a possible embodiment, the computer 1802 can be located remotely with respect to the image acquisition device 1814.

Any or all of the systems and apparatus discussed herein may be implemented using one or more computers such as computer 1802.

One skilled in the art will recognize that an implementation of an actual computer or computer system may have other structures and may contain other components as well, and that FIG. 18 is a high level representation of some of the components of such a computer for illustrative purposes.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

1. A computer-implemented method comprising:
receiving input medical data;
receiving results of a medical analysis task performed based on the input medical data using a machine learning based medical analysis network;
determining a robustness of the machine learning based medical analysis network for performing the medical analysis task based on the input medical data and the results of the medical analysis task using a machine learning based audit network; and
outputting the determination of the robustness of the machine learning based medical analysis network.

2. The computer-implemented method of claim 1, further comprising:
in response to determining that the machine learning based medical analysis network is not robust, determining that the machine learning based medical analysis network is not robust due to the input medical data being out-of-distribution with respect to training data on which the machine learning based medical analysis network was trained or due to an artifact in at least one of the input medical data or the results of the medical analysis task.

3. The computer-implemented method of claim 1, further comprising:
in response to determining that the machine learning based medical analysis network is not robust, retraining the machine learning based medical analysis network and the machine learning based audit network based on the input medical data.

4. The computer-implemented method of claim 1, further comprising:
in response to determining that the machine learning based medical analysis network is not robust, presenting one or more alternate results of the medical analysis task from other machine learning based medical analysis networks.

5. The computer-implemented method of claim 1, further comprising receiving user input editing the results of the medical analysis task to generate final results of the medical analysis task and wherein determining a robustness of the machine learning based medical analysis network for performing the medical analysis task based on the input medical data and the results of the medical analysis task using a machine learning based audit network comprises:
determining the robustness of the machine learning based medical analysis network based on the final results of the medical analysis tasks.

6. The computer-implemented method of claim 1, wherein the machine learning based audit network is implemented using a normalizing flows model.

7. The computer-implemented method of claim 1, further comprising:
in response to determining that the machine learning based medical analysis network is not robust, generating an alert to a user notifying the user that the machine learning based medical analysis network is not robust or requesting input from the user.

8. The computer-implemented method of claim 7, further comprising:
receiving the input from the user overriding the determination that the machine learning based medical analysis network is not robust or editing the results of the medical analysis task.

9. The computer-implemented method of claim 1, wherein the medical analysis task comprises at least one of segmentation, determining centerlines of vessels, or computing a fractional flow reserve (FFR).

10. An apparatus comprising:
means for receiving input medical data;
means for receiving results of a medical analysis task performed based on the input medical data using a machine learning based medical analysis network;
means for determining a robustness of the machine learning based medical analysis network for performing the medical analysis task based on the input medical data and the results of the medical analysis task using a machine learning based audit network; and
means for outputting the determination of the robustness of the machine learning based medical analysis network.

11. The apparatus of claim 10, further comprising:
means for determining that the machine learning based medical analysis network is not robust due to the input medical data being out-of-distribution with respect to training data on which the machine learning based medical analysis network was trained or due to an artifact in at least one of the input medical data or the results of the medical analysis task in response to determining that the machine learning based medical analysis network is not robust.

12. The apparatus of claim 10, further comprising:
means for retraining the machine learning based medical analysis network and the machine learning based audit network based on the input medical data in response to determining that the machine learning based medical analysis network is not robust.

13. The apparatus of claim 10, further comprising:
means for presenting one or more alternate results of the medical analysis task from other machine learning based medical analysis networks in response to determining that the machine learning based medical analysis network is not robust.

14. The apparatus of claim 10, further comprising means for receiving user input editing the results of the medical analysis task to generate final results of the medical analysis task and wherein the means for determining a robustness of the machine learning based medical analysis network for performing the medical analysis task based on the input medical data and the results of the medical analysis task using a machine learning based audit network comprises:
means for determining the robustness of the machine learning based medical analysis network based on the final results of the medical analysis tasks.

15. A non-transitory computer readable medium storing computer program instructions, the computer program instructions when executed by a processor cause the processor to perform operations comprising:
receiving input medical data;
receiving results of a medical analysis task performed based on the input medical data using a machine learning based medical analysis network;
determining a robustness of the machine learning based medical analysis network for performing the medical analysis task based on the input medical data and the results of the medical analysis task using a machine learning based audit network; and
outputting the determination of the robustness of the machine learning based medical analysis network.

16. The non-transitory computer readable medium of claim 15, wherein the machine learning based audit network is implemented using a normalizing flows model.

17. The non-transitory computer readable medium of claim 15, the operations further comprising receiving user input editing the results of the medical analysis task to generate final results of the medical analysis task and wherein determining a robustness of the machine learning based medical analysis network for performing the medical analysis task based on the input medical data and the results of the medical analysis task using a machine learning based audit network comprises:
determining the robustness of the machine learning based medical analysis network based on the final results of the medical analysis tasks.

18. The non-transitory computer readable medium of claim 15, the operations further comprising:
in response to determining that the machine learning based medical analysis network is not robust, generating an alert to a user notifying the user that the machine learning based medical analysis network is not robust or requesting input from the user.

19. The non-transitory computer readable medium of claim 18, the operations further comprising:
receiving the input from the user overriding the determination that the machine learning based medical analysis network is not robust or editing the results of the medical analysis task.

20. The non-transitory computer readable medium of claim 15, wherein the medical analysis task comprises at least one of segmentation, determining centerlines of vessels, or computing a fractional flow reserve (FFR).
